(19) United States
(12) Patent Application Publication
(10) Pub. No.: US 2011/0230935 A1
(43) Pub. Date: Sep. 22, 2011

(54) Rechargeable Stimulation Lead, System, and Method

(76) Inventor: Mark Zdeblick, Portola Valley, CA (US)

(21) Appl. No.: 12/601,171

(22) PCT Filed: Nov. 13, 2009
(86) PCT No.: PCT/US09/64440

§ 371 Date: Nov. 20, 2009

Related U.S. Application Data
(60) Provisional application No. 61/114,443, filed on Nov. 13, 2008.

(51) Int. Cl. A61N 1/36

(52) U.S. Cl. 607/59; 607/61

(57) ABSTRACT

Implantable electrical stimulation leads, method, and system are provided. Components of the system include a hermetically sealed integrated circuit controller, two or more hermetically sealed individually addressable satellite electrode structures and an inductive power source. The lead includes a housing, a conductor positioned within the housing, addressable stimulation units secured within the housing, wherein each stimulation unit includes a hermetically sealed integrated circuit, and a plurality of electrodes each electrically isolated from the other.

### RELATED APPLICATION AND CROSS REFERENCE

This application claims the benefit of U.S. Provisional Application Ser. No. 61/114,443 files on Nov. 13, 2008, the entire disclosure of which is incorporated herein by reference.

### FIELD OF THE INVENTION

The present invention is related to electrical devices and systems for stimulation of a target site and, more specifically, multiplexed rechargeable leads including multiple electrodes that are individually addressable and include an inductive power source and power storage units.

### BACKGROUND

Implantable neurostimulators are used to deliver neurostimulation therapy to patients to treat a variety of symptoms or conditions such as chronic pain, tremor, Parkinson's disease, epilepsy, incontinence, or gastroparesis. Implantable neurostimulators may deliver neurostimulation therapy in the form of electrical pulses via implantable leads that include electrodes. To treat the above-identified symptoms or conditions, implantable leads may be implanted along nerves, within the epidural or intrathecal space of the spinal column, and around the brain, or other organs or tissue of a patient, depending on the particular condition that is sought to be treated with the device.

With respect to implantable leads, several elements such as conductors, electrodes and insulators may be combined to produce a lead body. A lead may include one or more conductors extending the length of the lead body from a distal end to a proximal end of the lead. The conductors electrically connect one or more electrodes at the distal end to one or more connectors at the proximal end of the lead. The electrodes are designed to form an electrical connection or stimulus point with tissue or organs. Lead connectors (sometimes referred to as terminals, contacts, or contact electrodes) are adapted to electrically and mechanically connect leads to implantable pulse generators or RF receivers (stimulation sources), or other medical devices. An insulating material may form the lead body and surround the conductors for electrical isolation between the conductors and for protection from the external contact and compatibility with a body.

Such leads may be implanted into a body at an insertion site and extend from the implant site to the stimulation site (area of placement of the electrodes). The implant site may be a subcutaneous pocket that receives and houses the pulse generator or receiver (providing a stimulation source). The implant site may be positioned a distance away from the stimulation site, such as near the buttocks or other place in the torso area. One common configuration is to have the implant site and insertion site located in the lower back area, with the leads extending through the epidural space in the spine to the stimulation site, such as middle back, upper back, neck or brain areas.

Current lead designs have different shapes, such as those commonly known as paddle leads and percutaneous leads. Paddle leads, which are typically larger than percutaneous leads, are directional and often utilized due to desired stimulus effect on the tissues or areas. However, current paddle leads require insertion using surgical means, and hence, removal through surgical means. Percutaneous leads are designed for easy introduction into the epidural space using a special needle. Therefore, such leads are typically smaller and more nearly circular in cross-section than paddle-shaped leads. This reduced size facilitates their implantation in the body, allows their implantation into more areas of the body, and minimizes the unwanted side effects of their implantation.

### SUMMARY

Implantable electrical stimulation leads are provided. Components of the provided leads include a hermetically sealed integrated circuit controller, two or more hermetically sealed individually addressable satellite electrode structures and an inductive power source. Also provided are systems that include the leads of the invention, as well as methods of using the systems and leads in a variety of different applications.

### BRIEF DESCRIPTION OF THE FIGURES

FIG. 1 provides a view of a percutaneous lead according to one embodiment of the invention, where the percutaneous lead includes several individually addressable satellite electrode structures.

FIG. 1A provides an exploded view of an electrode structure of the lead of FIG. 1.

FIG. 2 provides a more detailed view of an individually addressable satellite electrode structure as may be present in a lead according to one aspect of the invention.

FIG. 3 provides a view of paddle lead according to another aspect of the invention.

FIG. 4 provides a view of a single electrode of the paddle lead of FIG. 3 according to the invention.

### DETAILED DESCRIPTION

Implantable electrical stimulation are configured for stimulating a variety of different types of tissue, including but not limited to nervous tissue, muscle tissue, etc. As such, they are structured for stimulation applications, in terms of device form factor or shape, as well as control unit programming. Devices of the invention may be configured for specific applications, such as neural stimulation applications. Such devices may have a variety of shapes that are suitable for use in neural stimulation applications, including shapes found in traditional percutaneous leads, paddle leads as well as other neurostimulation specific configurations. Programming (a set of instructions that are implemented by a processor to perform a given task) that is specific for a stimulation protocol of interest, such as a neural stimulation protocol or muscle tissue stimulation protocol may also be included in components of devices of the invention, such as integrated circuit elements of the integrated circuit controller and/or individually addressable satellite electrode structures of the devices, as reviewed in greater detail below. Programming that may be part of the devices may include a full set of instructions for a given task or a partial set of instructions that is employed in conjunction with other instructions associated with components distinct from the devices, where such additional instructions may be present in an extracorporeal control unit with which the device communicates at some time associated with operation of the device, e.g., before, during or after the device stimulates target tissue of interest.

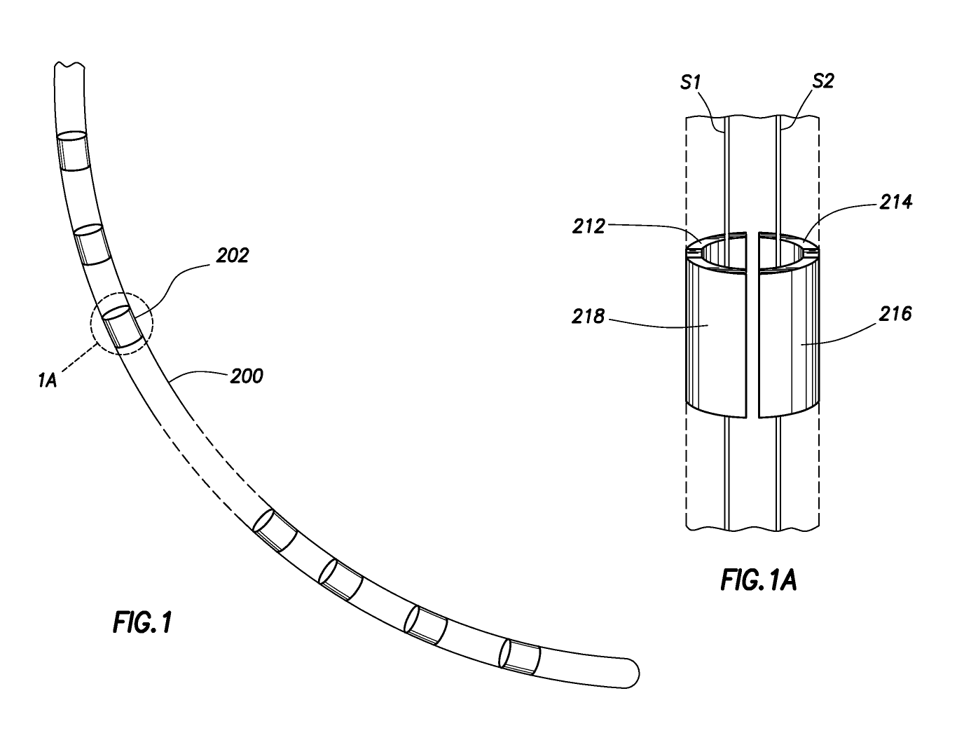

Referring now to FIG. 1 and FIG. 1A, a multiplexed multi-electrode lead 200 is shown. The lead 200 includes multiple individually addressable satellite structures 202 positioned longitudinally on the lead 200. The lead 200 includes two bus wires S1 and S2. The wires S1 and S2 are coupled to multiple structures 202, each being individually addressable. Referring now to FIG. 1A, one example of an individually addressable satellite electrode structure 202 is shown with multiple electrodes. In one aspect of the invention, structure 202 includes electrodes 212, 214, 216, and 218, located in the four quadrants of the cylindrical outer walls of structure 202. The scope of the invention is not limited by the number of electrode. An individually addressable satellite electrode structure of the present invention may include more or less than four electrode elements. Each individually addressable satellite electrode structure also contains an integrated circuit component inside the structure which communicates with other satellite structures and/or distinct control units, e.g., to receive stimulation signals and/or configuration signals that determine which of the different electrodes are to be coupled to bus wires S1 or S2.

As the lead 200 is implantable, it is configured to maintain functionality when present in a physiological environment, including a high salt, high humidity environment found inside of a body. Implantable devices of the invention are configured to maintain functionality under these conditions for two or more days, such as one week or longer, four weeks or longer, six months or longer, one year or longer, including five years or longer. In some instances, the implantable devices are configured to maintain functionality when implanted at a physiological site for a period ranging from one to eighty years or longer, such as from five to seventy years or longer, and including for a period ranging from ten to fifty years or longer.

The lead 200 includes a multiplexed configuration. By multiplexed configuration is meant that the integrated circuit control element is electrically coupled to the two or more individually addressable satellite electrode structures using a common conductor or conductors. As such, two or more of the individually addressable satellite electrode structures share a common conductor or set of conductors, as shown in FIG. 1A as S1 and S2, and do not have their own individual conductor or set of conductors linking them to the integrated circuit controller. The term “conductor” refers to a variety of configurations of electrically conductive elements, including wires, cables, etc. A variety of different structures may be implemented to provide the multiplex configuration. Multiplex configurations of interest include, but are not limited to, those described in: PCT application no. PCT/US2003/039524 published as WO 2004/052182; PCT application no. PCT/US2005/031559 published as WO 2006/029090; PCT application no. PCT/US2005/046811 published as WO 2006/069322; PCT application no. PCT/US2005/046815 published as WO 2006/069323; and PCT application no. PCT US2006/048944 published as WO 2007/075974; the disclosures of which are herein incorporated by reference.

Implantable electrical stimulation leads of the invention include a hermetically sealed integrated circuit controller and two or more hermetically sealed individually addressable satellite electrode structures. Integrated circuit components of the invention include the controller and two or more individually addressable satellite electrode structures. These components are constructs that include circuitry components and a solid support. The solid support may be small, for example where it is dimensioned to have a width ranging from 0.01 mm to 100 mm, such as from 0.1 mm to 20 mm, and including from 0.5 mm to 2 mm; a length ranging from 0.01 mm to 100 mm, such as from 0.1 mm to 20 mm, and including from 0.5 mm to 2 mm, and a height ranging from 0.01 mm to 10 mm, including from 0.05 mm to 2 mm, and including from 0.1 mm to 0.5 mm. The solid support element may take a variety of different configurations, such as but not limited to: a chip configuration, a cylinder configuration, a spherical configuration, a disc configuration, etc. A particular configuration may be selected based on intended application, method of manufacture, etc. While the material from which the solid support is fabricated may vary considerably depending on the particular device for which the device is configured for use, in certain instances the solid support is made up of a semiconductor material, such as silicon.

Integrated circuit components of the controllers and individually addressable satellite electrode structures may include a number of distinct functional blocks, i.e., modules. In some instances, the circuits include at least the following functional blocks: a power extraction functional block; an energy storage functional block; a sensor functional block; a communication functional block; and a device configuration functional block, etc.

Within a given controller or satellite electrode structure, at least some of, e.g., two or more, up to and including all of, the functional blocks may be present in a single integrated circuit. By single integrated circuit is meant a single circuit structure that includes all of the different desired functional blocks for the device. In these types of structures, the integrated circuit is a monolithic integrated circuit that is a miniaturized electronic circuit which may be made up of semiconductor and passive components that have been manufactured in the surface of a thin substrate of semiconductor material. Sensors of the invention may also include integrated circuits that are hybrid integrated circuits, which are miniaturized electronic circuits constructed of individual semiconductor devices, as well as passive components, bonded to a substrate or circuit board.

Integrated circuit controllers of the leads are integrated circuits that are configured to operate the satellite electrode structures, either alone or in conjunction with another device, such as an extracorporeal control unit. The integrated circuit controllers are configured to active the electrodes of the lead in a manner sufficient to sense electrical pulses and/or apply stimulation pulses as desired, e.g., to implement a particular stimulation program.

Satellite electrode structures are structures that include an integrated circuit control device and at least one electrode element. The satellite electrode structures of the invention include control circuitry in the form of an integrated circuit, examples of which are described above, that imparts addressability to the satellite electrode structure. The leads include two or more individually addressable satellite electrode structures. In some instances, more than two individually addressable satellite structures are present in the device, such as three or more, four or more, five or more, six or more, ten or more, twenty or more (including twenty-four), thirty or more, fifty or more, etc. Individually addressable satellite electrode structures are those that can be individually controlled by the integrated circuit controller, either alone or in conjunction with separate device, such as an extracorporeal control unit.

A given satellite electrode structure may include a single electrode element coupled to an integrated circuit, or two or more electrodes coupled to the same integrated circuit, such as three or more electrodes, four or more electrodes, six or more electrodes, etc. In various aspects, the structure includes two or more electrode elements, such as three or more electrode elements, including four or more electrode elements, e.g., where the structure is a segmented electrode structure. The various electrode elements may be positioned in three-dimensional space relative to their controlling integrated circuit to which they are electronically coupled in a number of different ways. For example, the multiple electrode elements may be radially distributed, i.e., axially uniformly positioned, about an integrated circuit. Alternatively, the multiple electrode elements may be positioned to one side of an integrated circuit.

In some instances, the sealing element is a conformal, void-free sealing layer, where the sealing layer is present on at least a portion of the outer surface of the integrated circuit component (described above). In some instances, this conformal, void-free sealing layer may be present on substantially all of the outer surfaces of the integrated circuit component. Alternatively, this conformal, void-free sealing layer may be present on only some of the surfaces of the integrated circuit, such as on only one surface or even just a portion of one surface of the integrated circuit component. As such, some sensors have an integrated circuit component completely encased in a conformal, void free sealing layer. Other sensors are configured such that only the top surface of an integrated circuit component is covered with the conformal, void-free sealing layer.

The conformal, void-free sealing layer may be a “thin-film” coating, in that its thickness is such that it does not substantially increase the total volume of the integrated circuit structure with which it is associated, where any increase in volume of the structure that can be attributed to the layer may be 10% or less, such as 5% or less, including 1% or less by volume. In some instances, the seal layer has a thickness in a range from 0.1 to 10.0 μm, such as in a range from 0.3 to 3.0 μm thick, and including in a range 1.0 μm thick.

The seal layer may be produced on the integrated circuit component using any of a number of different protocols, including but not limited to planar processing protocols, such as plasma-enhanced-chemical-vapor deposition, physical-vapor deposition, sputtering, evaporation, cathodic-arc deposition, low-pressure chemical-vapor deposition, etc.

Additional description of conformal, void-free sealing layers that may be employed for sensors of the invention is provided in PCT application serial no. PCT/US2007/009270 published under publication no. WO/2007/120884, the disclosure of which is herein incorporated by reference.

Also of interest as sealing elements are corrosion-resistant holders having at least one conductive feed-through and a sealing layer; where the sealing layer and holder are configured to define a hermetically sealed container that encloses the integrated circuit component. The conductive feed-through may be a metal, such as platinum, iridium etc., an alloy of metal and a semiconductor, a nitride, a semiconductor or some other convenient material. In some instances, the corrosion-resistant holder comprises silicon or a ceramic. While dimensions may vary, the corrosion-resistant holder may have walls that are at least 1 μm thick, such as at least 50 μm thick, where the walls may range from 1 to 125 μm, including from 25 to 100 μm. The sealing layer may be metallic, where metals of interest include noble metals and alloys thereof, such as platinum and platinum alloys. Dimensions of the sealing layer may also vary, ranging in some instances from 0.5 μm thick or thicker, such as 2.0 μm thick or thicker, and including 20 μm thick or thickness, where sealing layer thicknesses may range from 0.5 to 100 μm, such as from 1 to 50 μm. In certain configurations, the structure further includes an insulative material present in the hermetically sealed volume. In some cases, the hermetically sealed volume ranges from 1 pl. to 1 ml.

In some instances, the in-vivo corrosion-resistant holder is a structure configured to hold an integrated circuit component such that the integrated circuit component is bounded on all but one side by the walls of the holder. For example, the holder may include side walls and a bottom, where the holder may have a variety of different configurations as long as it contains the integrated circuit component in a manner such that the component is held in a volume bounded on all but one side. Accordingly, the shape of the holder may be square, circular, ovoid, rectangular, or some other shape as desired.

Additional description of corrosion resistant holders that may be employed for sensors of the invention is provided in PCT application serial no. PCT/US2005/046815 published under publication no. WO/2006/069323, the disclosure of which is herein incorporated by reference.

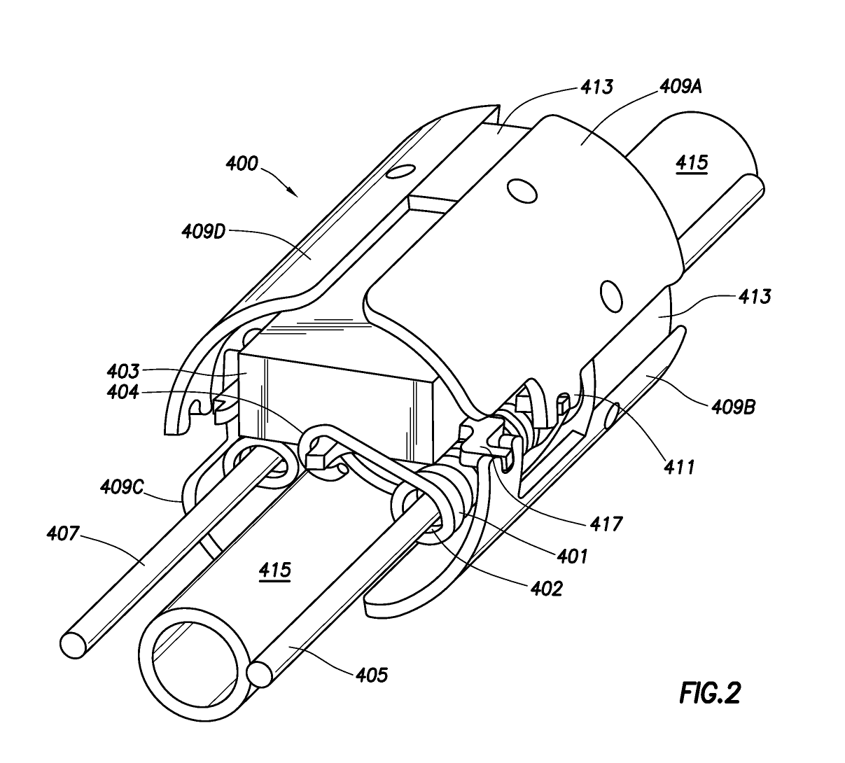

FIG. 2 shows a detailed view of one embodiment of an individually addressable segmented electrode structure 400. The structure 400 has four electrode elements 409A, 409B, 409C, and 409D radially positioned about a hermetically sealed integrated circuit component. The configuration may be viewed as a quadrant electrode. Flexible connections 401 are provided between element 403 and elongated conductive members 405 and 407, for example S1 and S2 of FIG. 1. According to various aspects of the invention, the element 403 may be an integrated circuit or it may be a housing that includes multiple components such as an integrated circuit as a power storage unit. This design creates a flexible connection between the integrated circuit and the elongated conductive members. Each of the flexible connections 401 include a securing hook 404 for securely holding the element 403 in place. As shown, the elongated conductive members 405 and 407 are placed into inner lumen 402 of flexible connections 401. Element 403 is attached to the four distinct electrodes 409A, 409B, 409C and 409D by junctures 411 and 417. Electrodes 409A, 409B, 409C and 409D are joined together in a suitable configuration structure 413, which may be made of any convenient material, such as polyetheretherketone (PEEK). Guide wire lumen 415 runs beneath element 403 and beneath and/or between elongated conductive members 405 and 407, all running through or contained within the area defined by the position and orientation of the electrodes 409A, 409B, 409C and 409D.

Element 403 of the satellite electrode structures 400 that are within the lead 200, of FIG. 1, have hermetically sealed integrated circuit components, such that they include a sealing element which seals the integrated circuit component from the implanted environment so that the integrated circuit component maintains functionality, at least for the intended lifespan of the device. The nature of the sealing element may vary, so long as it maintains the functionality of the component in the implanted environment for the desired period of time, such as one week or longer, one month or longer, one year or longer, five years or longer, ten years or longer, twenty-five years or longer, forty years or longer.

Additional details regarding individually addressable satellite electrode structures can be found in PCT application serial no. PCT/US2005/031559 published as WO 2006/029090; PCT application serial no. PCT/US2005/046815 published as WO 2006/069323; PCT application serial no. PCT/US2005/046811 published as WO 2006/069322; and U.S. application Ser. No. 11/939,524 published as US 2008-0114230 A1; the disclosures of which are herein incorporated by reference.

In addition to the hermetically sealed integrated circuit controller and individually addressable satellite electrode structures, leads of the invention may also include an inductive power source. The inductive power source is a component configured to receive power signals from an extracorporeal location, e.g., in the form of radiofrequency energy, and convert the received signals into energy sufficient to power the lead. The inductive power source may take any convenient shape. In some instances, the inductive power source is a coil. Coils employed in inductive power sources of the leads may vary, from loose coils to tight coils, as desired depending on the particular lead configuration. The inductive power sources may be positioned at any convenient location in the lead, including in the center of the lead, on the periphery of the lead, etc.

Where desired, leads of the invention may further include an energy storage component. Energy storage components of interest are structures that are capable of storing the energy provided by the inductive power source for use at a later time. Any convenient energy storage component may be employed, including but not limited to capacitors, batteries, etc. Leads including energy storage components may be viewed as rechargeable.

Lead components are elongated structures having lengths that are 2 times or longer than their widths, such as 5 times or longer than their widths, including 10, 15, 20, 25, 50, 100 times or longer than their widths. In certain instances, the leads have lengths of 10 mm or longer, such as 25 mm or longer, including 50 mm or longer, such as 100 mm or longer. A variety of different lead configurations may be employed. The lead may include one or more lumens, e.g., for use with a guidewire, for housing one or more conductive elements, e.g., wires, etc. The distal end may include a variety of different features as desired, e.g., a securing means, etc. Leads may be fabricated as flexible structures, where internal conductor elements may include wires, coils or cables made of a suitable material, such as alloy MP35N (a nickel-cobalt-chromium-molybdenum alloy), platinum, platinum-10 iridium, etc. The lead body may be any suitable material, such as a polymeric material, including polyurethane or silicone.

Lead components of the invention may have a variety of shapes, as desired. In some instances, the leads have a standard percutaneous shape, as found in conventional percutaneous neural stimulation leads, for example an elongated cylindrical or other structure configured to be positioned in the epidural space. In some instances, the leads have a standard paddle shape, as found in conventional paddle neural stimulation leads, where the electrodes are displayed in a two-dimensional array.

Leads of the invention are configured to communicate with a distinct device, such as a control unit, that is not physically coupled to the lead. This distinct device is one that may be implanted in a subject or extracorporeal, as desired. However, because the device is not physically coupled to the lead, the lead is not joined by a physical structure to the distinct device. Accordingly, the lead is one that is configured not to be physically coupled to a distinct device. Therefore, the lead includes no component that would provide for physical connection to a distinct device, such as an IS-1 connector.

FIG. 3 provides a view of a paddle lead 300 according to the invention, where the paddle lead 300 is a rechargeable device that is not configured to be coupled to any other component, such as an implantable control unit, e.g., an implantable pulse generator. The paddle lead 300 is a standalone lead that nonetheless can provide stimulation to target tissue without being physically coupled to a distinct control unit, such as an implantable pulse generator. The paddle lead 300 includes flexible lead support 310 that includes a conventional paddle lead shape. On one surface of the paddle lead 300 is a two-dimensional array of electrodes 320, which are electrodes of individually addressable satellite electrode structures (as described above). Also shown is a hermetically sealed integrated circuit controller 330 which is coupled to the individually addressable satellite electrodes by a multiplex configuration. Paddle lead 300 also includes a coil 340 made up of a wire that is wrapped two or more times about the periphery of the paddle. Also shown is energy storage element 350 which may be a capacitor or battery.

Any of a variety of different protocols may be employed in manufacturing the devices of the invention. For example, molding, deposition and material removal, planar processing techniques, such as Micro-Electro-Mechanical Systems (MEMS) fabrication, may be employed. Deposition techniques that may be employed in certain aspects of fabrication of the devices or components thereof include, but are not limited to: electroplating, cathodic arc deposition, plasma spray, sputtering, e-beam evaporation, physical vapor deposition, chemical vapor deposition, plasma enhanced chemical vapor deposition, etc. Material removal techniques of interest include, but are not limited to: reactive ion etching, anisotropic chemical etching, isotropic chemical etching, planarization, e.g., via chemical mechanical polishing, laser ablation, electronic discharge machining (EDM), etc. Also of interest are lithographic protocols. Of interest in certain embodiments is the use of planar processing protocols, in which structures are built up and/or removed from a surface or surfaces of an initially planar substrate using a variety of different material removal and deposition protocols applied to the substrate in a sequential manner.

In some instances, laser cut wires are employed as conductive elements for devices of the invention, such as for conductive elements of lead elements of devices of the invention. For example, conductive elements may be laser cut from a single sheet of metal. The pattern of the laser cut conductive elements may be chosen to match the positioning of the individually addressable satellite electrode structures of the lead. In this manner, the conductors and electrode structures may be aligned and then overlaid with the appropriate polymeric material to produce the desired lead structure. The laser cut conductive elements may have a variety of configurations from linear to curvilinear, sinusoidal or other fatigue resistance configurations. Instead of laser cutting, the conductor could also be fabricated using a deposition protocol, such as described above.

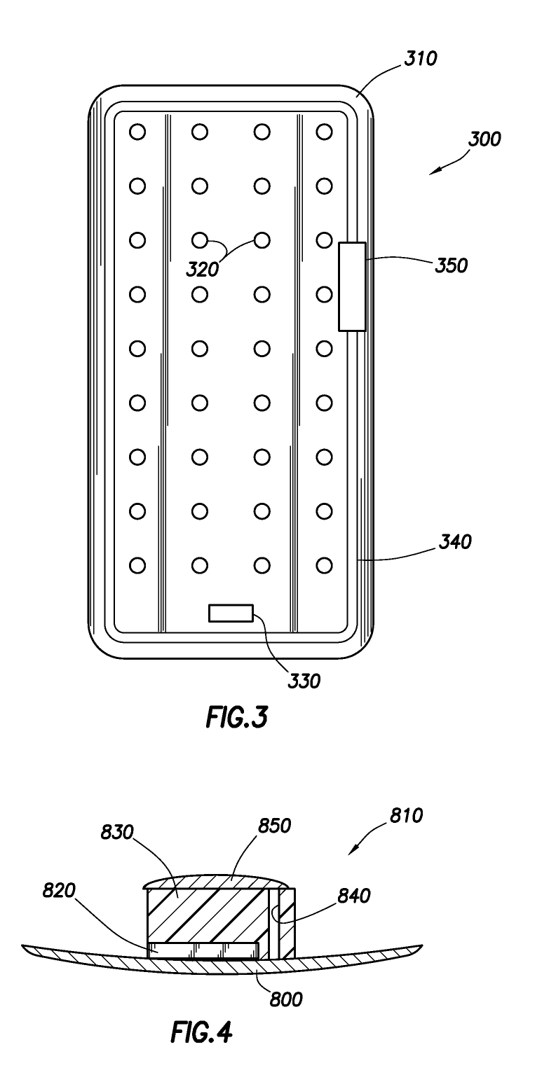

FIG. 4 provides a cross-section view of a laser cut conductive element 800 of an individually addressable satellite electrode structure 810. Integrated circuit component 820 is in electrical contact with the laser cut conductive element 800. Conformal void free layer 830 is present on top of the integrated circuit component 820 and, upon overlay of the polymeric coating, hermetically seals the integrated circuit component 820. A deposited single electrode 850 is present on top of the layer 830 and connected to integrated circuit component 820 via connector 840. The electrode 850 may be deposited using any convenient protocol, such as cathodic arc deposition.

The electrode structure 810 shown in FIG. 4 may be used in the paddle lead 300 of FIG. 3. For such a lead, underlying the shown electrodes 320, which are similar to electrode structures 810, may be a laser cut pattern of conductive elements as described above. All of the electrodes 320 of the paddle lead 300 are coupled to the laser cut pattern of conductive elements to provide electrical communication between the electrodes and integrated circuit controller.

Devices of the invention may be implanted using any convenient protocol. Standard implantation procedures for percutaneous and paddle leads may be adapted for implantation of devices of the invention. The devices may be configured for ease of implantation. For example, devices may include a deployable element, such as lead components that inflate, e.g., with a gas or suitable liquid medium, to assume a desired configuration.

Also provided are systems that include one more neural stimulation devices as described in communication with a distinct controller, e.g., an implantable such as an implantable pulse generator or an extracorporeal controller, such as one that is configured to transmit data and/or power to and/or receive data from the implantable components.

Also provided are methods of using the systems of the invention. The methods of the invention may include: providing a system of the invention that includes an implantable electrical stimulation lead of the invention, as described above. The lead may be implanted in a suitable subject using any convenient approach. Following implantation, the lead may be employed to as desired to treat a condition of interest.

During use, a health care professional, such as a physician or other clinician, may select values for a number of programmable parameters in order to define the neurostimulation therapy to be delivered to a patient. For example, the health care professional may select a voltage or current amplitude and pulse width for a stimulation waveform to be delivered to the patient, as well as a rate at which the pulses are to be delivered to the patient and a duty cycle. The health care professional may also select as parameters particular electrodes within the electrode set carried by the leads to be used to deliver the pulses, and the polarities of the selected electrodes. A group of parameter values may be referred to as a program in the sense that they drive the neurostimulation therapy to be delivered to the patient.

A health care professional may select parameter values for a number of programs to be tested on a patient during a programming session. The programming device directs the implantable neurostimulator implanted in the patent to deliver neurostimulation according to each program, and the health care professional collects feedback from the patient, e.g., rating information, for each program tested on the patient. The health care professional then selects one or more programs for long-term use by the implantable neurostimulator based on the rating information.

Implantable stimulation devices of the invention find use in any application where electrical stimulation of target tissue in a patient is desired. Implantable neurostimulator devices of the invention may be employed in a variety of different applications. Examples of applications include the use of the devices and systems to deliver neurostimulation therapy to patients to treat a variety of symptoms or conditions such as chronic pain, tremor, Parkinson's disease, epilepsy, incontinence, or gastroparesis. Implantable neurostimulators may deliver neurostimulation therapy in the form of electrical pulses via leads that include electrodes. To treat the above-identified symptoms or conditions, for example, the electrodes may be located proximate to the spinal cord, pelvic nerves, or stomach, or within the brain of a patient.

Also provided are kits that include the devices or components therefore, e.g., leads and controllers, etc. In various aspects of the subject kits, the kits will further include instructions for using the subject devices or elements for obtaining the same (e.g., a website URL directing the user to a webpage which provides the instructions), where these instructions are typically printed on a substrate, which substrate may be one or more of: a package insert, the packaging, reagent containers and the like. In the subject kits, the one or more components are present in the same or different containers, as may be convenient or desirable.

It is to be understood that this invention is not limited to particular embodiments described, as such may vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

What is claimed is:

1. A device for providing electrical stimulation to various target sites, the device comprising:
a housing including a guide positioned in the housing and continuing at least a portion of the length of the housing;
at least one conductor positioned within the housing and continuing through at least a portion of the length of the housing; and
a plurality of addressable stimulation units secured within the housing, wherein each stimulation unit comprises:
a hermetically sealed integrated circuit electrically coupled to the conductor for receiving power and secured to the guide; and
a plurality of electrodes each electrically isolated from the other and each electrically coupled to the circuit, such that the integrated circuit can control the supply of power to each electrode independent of other electrodes,
wherein the lead is placed near the various target sites such that each stimulation unit is positioned at one target site and electrical stimulation of each target site is controlled by the integrated circuit of the stimulation unit located proximal to that specific target site.

2. The device of claim 1 further comprising a second conductor positioned within the housing and continuing through at least a portion of length of the housing and electrically coupled to the integrated circuit.

3. The device of claim 2 further comprising an inductive power source positioned at one end of the device and electrically coupled to the conductor and the second conductor wherein the coil represents an inductive power source.

4. The device of claim 3 claim 1 further comprising an energy storage component positioned at one end of the device and electrically coupled to the inductive power source.

5. The device of claim 4 wherein the energy storage component is a battery.

6. The device of claim 4 wherein the energy storage component is a capacitor.

7. The device of claim 3 wherein the inductive power source is a coil.

8. The device of claim 1 further comprising an inductive power source positioned at one end of the device such that a first connection of the coil is electrically coupled to the environment of the various target sites and a second connection of the coil is electrically coupled to the conductor and wherein the opposite end of the conductor is also electrically coupled to the environment of the various target sites to complete the conduction path.

9. The device of claim 1 wherein the lead has a paddle configuration with the stimulation units arranged in a two dimensional array.

10. The device of claim 9 further comprising an inductive power source and wherein the coil of the lead comprises a conductor wrapped two or more times about the periphery of the lead.

11. The device of claim 1 wherein each integrated circuit is configured to communicate with an extracorporeal control unit for programming the integrated circuit.

12. The device of claim 1 wherein each stimulation unit is positioned at a selected distance from nearby stimulation units.

13. The device of claim 12 wherein each stimulation unit conforms to the shape of the housing.

14. A tissue stimulation system comprising:
an extracorporeal control unit; and
an implantable electrical stimulation lead comprising:
a conductor continuing along at least a portion of the lead;
a hermetically sealed integrated circuit controller electrically coupled to the conductor and positioned at one end of the lead;
a plurality of addressable satellite electrode structures each electrically coupled to the conductor and controlled by the integrated circuit controller; and
an inductive power source coupled to the integrated circuit controller for supplying inductive power to the electrode structures,
wherein the extracorporeal control unit is configured to transmit programming to the implantable electrical stimulation lead.

15. The system of claim 12 wherein the system further comprises a power storage unit coupled to the conductor of the lead for supplying power to the electrode structures through the integrated circuit controller.
